(19) United States
(12) Patent Application Publication
(10) Pub. No.: US 2026/0108384 A1
(43) Pub. Date: Apr. 23, 2026

(54) SYSTEM AND METHOD FOR COOLING A PERSON INCORPORATING HEART RATE VARIABILITY MONITORING SENSOR

(71) Applicant: TecTraum, Inc., Hudson, OH (US)

(72) Inventors: John F. Zak, III, Chagrin Falls, OH (US); William Stuart, Mentor, OH (US)

(21) Appl. No.: 19/117,727

(22) PCT Filed: Mar. 7, 2023
(86) PCT No.: PCT/US2023/014685

§ 371 Date: Apr. 2, 2025

Related U.S. Application Data
(60) Provisional application No. 63/413,343, filed on Oct. 5, 2022.

(51) Int. Cl. A61F 7/00

(52) U.S. Cl. CPC A61F 7/0085; A61F 7/007; A61F 2007/0007; A61F 2007/0009; A61F 2007/0035; A61F 2007/0056; A61F 2007/0078; A61F 2007/0086; A61F 2007/0093

(57) ABSTRACT

A system for cooling a person includes a pump, a heat exchanger, a bladder, a thermometer, a heart rate variability (HRV) sensor, and a controller. The heat exchanger is in fluid communication with the pump. The bladder is configured to be placed on a person, and is in fluid communication with the heat exchanger. The thermometer measures a temperature of fluid passing through at least one of the pump, the heat exchanger and the bladder. The controller is in electrical communication with the thermometer, the HRV sensor and the heat exchanger, and is configured to control power delivered to or flow through the heat exchanger based on signals received from the HRV sensor.

### BACKGROUND

U.S. Pat No. 6,183,501 B1 discloses a cooling system having a head and neck device which can be cooled to reduce trauma to the brain. The head device includes panels that each house a cold element to facilitate cooling. The head device secures to the head of an individual and covers over the individual's carotid arteries, which provide blood to the brain. US 2012/0288848 A1 discloses similar devices connected with a pump and cooling fluid source.

US 2021/0128345 A1 discloses a system for treating a brain injury that includes a pump, a heat exchanger, a bladder, a thermometer, and a controller. The heat exchanger is in fluid communication with the pump. The bladder is configured to be placed over a carotid artery, and is in fluid communication with the heat exchanger. The thermometer is located with respect to the heat exchanger and configured to measure a temperature of fluid downstream from the heat exchanger. The controller is in electrical communication with the thermometer and the heat exchanger. The controller is configured to control power delivered to or flow through the heat exchanger such that the temperature of the fluid downstream from the heat exchanger measured by the thermometer is between 2 degrees C and 10 degrees C for between 10 minutes and 50 minutes. The system is configured to address comfort issues that may arise when an individual wears such a head cooling device.

Further control of the magnitude of cooling and/or duration of cooling based on physiological measurements obtained from the person being treated can be useful to further tailor treatment. Moreover, these measurements can be useful whether treating a person for a brain injury or simply when cooling the person, for example a person undergoing strenuous physical activity.

### SUMMARY

A system for cooling a person includes a pump, a heat exchanger, a bladder, a thermometer, a heart rate variability (HRV) sensor, and a controller. The heat exchanger is in fluid communication with the pump. The bladder is configured to be placed on a person, and is in fluid communication with the heat exchanger. The thermometer measures a temperature of fluid passing through at least one of the pump, the heat exchanger and the bladder. The controller is in electrical communication with the thermometer, the HRV sensor and the heat exchanger, and is configured to control power delivered to or flow through the heat exchanger based on signals received from the HRV sensor.

A method for cooling a person includes pumping fluid through a heat exchanger to a bladder placed on a person, and removing heat from the fluid as the fluid passes through the heat exchanger. The method further includes measuring a temperature of the fluid passing through the heat exchanger, monitoring the person's HRV with an HRV sensor in communication with a controller, and controlling at least one of power delivered to the heat exchanger and flow of fluid through the heat exchanger based on signals received from the HRV sensor.

### BRIEF DESCRIPTION OF THE DRAWINGS

FIG. 1 is a schematic depiction of a system for cooling a person.

FIG. 2 is a perspective view of bladders and carriers of the system depicted in FIG. 1.

FIG. 3 is a schematic depiction of a chiller unit of the system depicted in FIG. 2.

FIG. 4 is a flow chart depicting an example of a method of operating the system depicted in FIG. 1.

### DETAILED DESCRIPTION

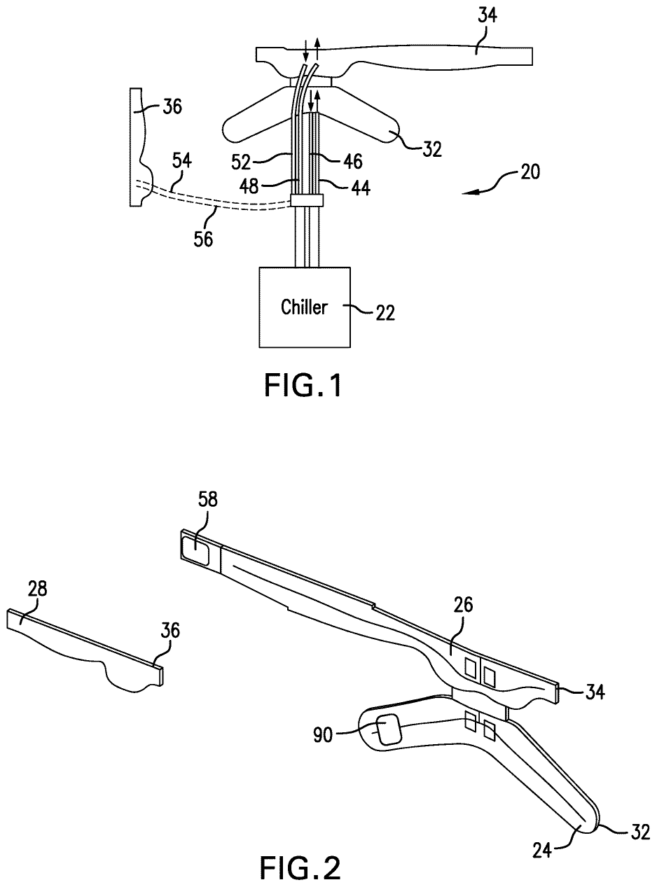

FIG. 1 depicts a system 20 that is useful in cooling a person whether for treating a brain injury or for simply cooling the person. The system 20 generally includes a chiller unit 22 connected with bladders 24, 26, 28 (FIG. 2) carried by respective carriers 32, 34, 36 via fluid lines 44, 46, 48, 52, 54, 56, respectively. With reference to FIG. 2, the lower bladder 24 is configured to be placed around the neck 14 and over a person's carotid arteries. The upper bladder 26 can be wrapped around the forehead region 12. The third bladder 28 can be wrapped around a person's wrist, for example, and other bladders (not shown) can be provided and connected with the chiller unit 22 in a similar manner. The bladders 24, 26, 28 can be retained against the person using hook and loop fasteners 58, only one example of which is shown in FIG. 2. Cool fluid from the chiller unit 22 is pumped to the bladders 24, 26, 28 to cool blood flowing through the arteries in a manner that provides brain cooling and also provides cooling in general.

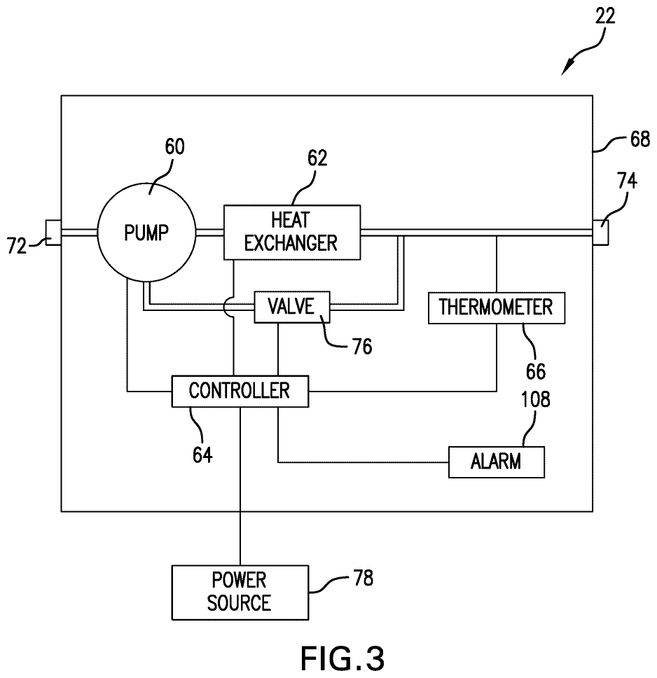

FIG. 3 schematically depicts the chiller unit 22. The chiller unit 22 includes a pump 60, a heat exchanger 62, a controller 64, and a thermometer 66. The pump 60, the heat exchanger 62, the controller 64, and the thermometer 66 are disposed in a casing 68, which is schematically depicted in FIG. 3. The chiller unit 22 includes a chiller inlet 72, which receives relatively warmer fluid from the bladders 24, 26, 28. The pump 60 moves the fluid incoming from the chiller inlet 72 through the heat exchanger 62 where the fluid can be cooled to a desired temperature and then pumped through a chiller outlet 74 back toward the bladders 24, 26, 28. If desired, the heat exchanger 62 can be operated to heat fluid. The chiller unit 22 can also include a valve 76, which when open can allow fluid from the pump 60 to bypass the heat exchanger 62. The valve 76 is configured to open to allow fluid to bypass the heat exchanger 62. The valve 76 can be operable in different operating states, e.g., fully open, fully closed and different states of being partially opened. When fully closed, the valve 76 does not allow fluid to pass through it. Upon receiving signals from the controller 64, the valve 76 can be operated similar to a throttle between fully open and fully closed to control the volume of fluid per unit of time flowing through the valve 76 and bypassing the heat exchanger 62.

The chiller unit 22 in the illustrated embodiment receives power from an external power source 78, which can provide power to each of the components of the chiller unit 22. The power source 78 could also be located within the casing 68, for example when the power source is a battery or battery pack. Positioning the power source 78 on or within the casing 68 can be desirable when reducing the size of the chiller unit is desired to make the chiller unit 22 portable so that it can be worn by the user, e.g., placed in a backpack, while the user, for example, is undergoing strenuous physical activity. The chiller unit 22 can also include a display and a user interface, which are not shown, to allow an operator to operate the chiller unit 22.

The thermometer 66 is located with respect to the pump 60, the heat exchanger 62 and the bladders 24, 26, 28 to measure a temperature of fluid passing through at least one of the pump 60, the heat exchanger 62 and the bladders 24, 26, 28. As depicted in FIG. 3, the thermometer 66 can measure the temperature of the fluid exiting the heat exchanger 62 prior to the fluid exiting the casing 68 and entering the fluid lines 44, 48, 54 respectively. Alternatively, the thermometer 66 can be located elsewhere, e.g., on one of the bladders 24, 26, 28, and can be in communication with the controller 64 via a wired or wireless connection, e.g. Bluetooth or other short-range wireless transmission protocol. The thermometer 66 communicates with the controller 64 to provide the controller the measured temperature of the fluid exiting the heat exchanger 62. Based on the measured temperature, the controller 64 can adjust the power, for example by using pulse width modulation (PWM), delivered to the heat exchanger 62. More power can be delivered to the cooling side of the heat exchanger 62 when the measured temperature is higher than the desired temperature. Power can be delivered to the heating side of the heat exchanger 62 when the measured temperature is lower than the desired temperature. In addition or alternatively to controlling power to the heat exchanger 62, the controller 64 can open and close the valve 76. For example, the valve 76 can be opened and fluid allowed to bypass the heat exchanger 62 in route to the chiller outlet 74 when the measured temperature is lower than a desired temperature. By way of example, when the thermometer 66 measures the temperature of the fluid exiting the heat exchanger as too cold (based on a predetermined threshold), then the controller 64 can open the valve 76 to allow relatively warmer fluid from upstream of the heat exchanger 62 to bypass the heat exchanger to raise the temperature of the fluid being delivered to the chiller outlet 74. Alternatively, the flow rate of the pump 60 can be adjusted, e.g., lowered, so that less fluid is delivered to the heat exchanger 62 when the measured temperature is lower than the desired temperature.

While heart rate focuses on average heartbeats per minute, heart rate variability (HRV) measures the specific changes in time (or variability) between successive heartbeats. A person's heart beats at a specific rate at all times. That rate changes depending on what the person is doing at the time. Slower heart rates happen when resting or relaxing, and faster rates happen when a person is active, stressed or in danger. There is variability in a person's heart rate based on the needs of one's body and respiratory patterns. Certain medications and medical devices can also affect heart rate variability. FIG. 2 depicts a heart rate variability (HRV) sensor 90 provided on the lower carrier 32, which typically wraps around a wearer's neck. However, it is to be understood that the HRV sensor 90 could be mounted on the other carriers 34, 36 or worn separately, e.g., not connected with a carrier. The HRV sensor 90 is in communication with the controller 64, which in the illustrated embodiment is via a wired or wireless connection, e.g. Bluetooth or other short-range wireless transmission protocol.

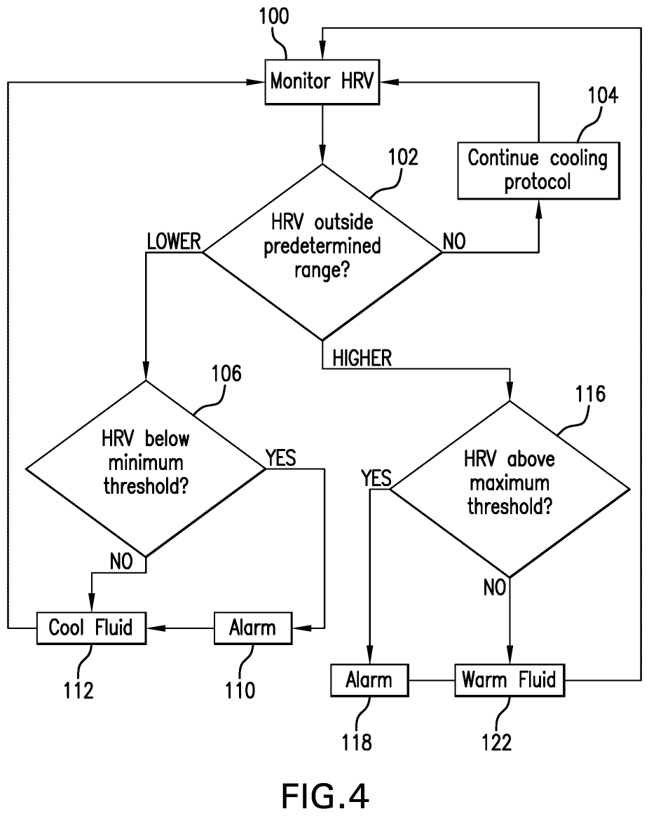

FIG. 4 depicts a particular example of a method of operating the system 20 that is useful in cooling a person using the HRV sensor 90 depicted in FIG. 2. Although FIG. 4 may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps. FIG. 4 is just one example of such a method and should not be found to limit the invention, which is defined by the claims.

At 100, the patient's HRV is monitored, for example, via the HRV sensor 90 (FIG. 2) communicating with the controller 64 (FIG. 3). The controller 64 can run a program that, at 102 (FIG. 4), determines whether the measured HRV is outside a predetermined range, which can be defined by a lower threshold and an upper threshold. For example, an HRV measured between 50 milliseconds (ms) and 150 ms may be considered “normal,” whereby measured HRV within this range may be considered not to be outside the predetermined range. When it is determined that the HRV measurement is not outside this predetermined range, then, at 104, the cooling protocol such as the one described in US 2021/0128345 A1, may be continued and the HRV can be continually monitored at 100. US 2021/0128345 A1, which is incorporated by reference herein, describes power delivered to or flow through the heat exchanger 62 being controlled such that the measured temperature of the fluid exiting the heat exchanger 62 is a treatment temperature between 2° C. and 10° C. (e.g., 6° C.) for a desired treatment time period, e.g., about 30 minutes. Other cooling protocols could be employed, an example being that fluid circulating through the bladders 24, 26, 28 is maintained around 6° C. (or other desired temperature) while the person wearing the bladders is undergoing strenuous physical activity.

If at 102, it is determined that the HRV is outside the predetermined range and lower than the lower threshold, then at 106, a determination is made whether the measured HRV is below a minimum threshold. For example, if at 106 it is determined that the HRV is below a minimum threshold, which for example could be set at 20 ms, then an alarm 108 (FIG. 3) can be set off at 110, and the fluid exiting the casing 68 and entering the fluid lines 44, 48, 54 leading to the bladders 24, 26, 28 can be further cooled, at 112. The further cooling may only be to a lower temperature threshold, e.g., 2° C. The alarm 108 can be in communication with the controller 64 and be provided on the casing 68 to provide a visual or audible indication to the wearer or caregiver that the wearer's HRV is dangerously low. If, however, at 106, it is determined that the HRV is above the minimum threshold, e.g., the measured HRV is still below the lower threshold of the predetermined range but above the minimum threshold (for example, 21-49 ms), then the fluid exiting the casing 68 and entering the fluid lines 44, 48, 54 can be further cooled at 112 without setting the alarm.

The fluid can be further cooled at 112 by adjusting the power delivered to the heat exchanger 62 so that more power is delivered to the cooling side of the heat exchanger 62. The method of operating the system 20 can then return to monitoring the patient's HRV at 100.

If at 102, it is determined that the HRV is outside the predetermined range and is higher than the upper threshold, then at 116, a determination is made whether the measured HRV is above a maximum threshold. For example, if at 116 it is determined that the HRV is above the maximum threshold, which for example could be set at 200 ms, then the alarm 108 can be set off at 118, and the fluid exiting the casing 68 and entering the fluid lines 44, 48, 54 can be warmed, at 122. The alarm 108 can also provide a visual or audible indication to the wearer or caregiver that the wearer's HRV is dangerously high. If, however, at 116, it is determined that the HRV is below the maximum threshold, e.g., the measured HRV is still above the upper threshold of the predetermined range but below the maximum threshold (for example, 151-200 ms), then the fluid exiting the casing 68 and entering the fluid lines 44, 48, 54 leading to the bladders 24, 26, 28 can be warmed at 122 without setting the alarm 108.

The fluid can be warmed by adjusting the power delivered to the heat exchanger 62 so that less power is delivered to the cooling side of the heat exchanger 62, by delivering power to a heating side of the heat exchanger 62, and/or by opening the valve 76 so that fluid bypasses the heat exchanger 62 to allow relatively warmer fluid from upstream of the heat exchanger 62 to bypass the heat exchanger to raise the temperature of the fluid being delivered to the chiller outlet 74. The fluid can also be warmed through the controller 64 controlling the pump 60 to control the flow rate through the pump 60, e.g., the flow rate can be slowed. The method of operating the system 20 can then return to monitoring the patient's HRV at 100.

Even though the invention has been described with reference to specific exemplifying embodiments thereof, many different alterations, modifications and the like will become apparent for those skilled in the art. Variations to the disclosed embodiments can be understood and effected by the skilled artisan in practicing the claimed invention, from a study of the drawings, the disclosure, and the appended claims. Furthermore, in the claims, the word “comprising” does not exclude other elements or steps, and the indefinite article “a” or “an” does not exclude a plurality. It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A system for cooling a person, comprising:
a pump;
a heat exchanger in fluid communication with the pump;
a bladder configured to be placed on a person, the bladder being in fluid communication with the heat exchanger;
a thermometer is located with respect to the pump, the heat exchanger and the bladder to measure a temperature of fluid passing through at least one of the pump, the heat exchanger and the bladder;
a heart rate variability (HRV) sensor; and
a controller in electrical communication with the thermometer, the HRV sensor and the heat exchanger, the controller configured to control power delivered to or flow through the heat exchanger based on signals received from the HRV sensor.

2. The system of claim 1, wherein the controller is configured to determine whether a measured HRV is outside a predetermined range defined by a lower threshold and an upper threshold.

3. The system of claim 2, wherein the controller is further configured to determine whether the measured HRV is below a minimum threshold while or after determining the measured HRV is outside the predetermined range and below the lower threshold.

4. The system of claim 3 further comprising an alarm in electrical communication the controller, and the controller is further configured to set off the alarm upon determining the measured HRV is outside the predetermined range and below the minimum threshold.

5. The system of claim 3, wherein the controller is further configured to control the heat exchanger to further cool fluid entering the bladder upon determining the measured HRV is below the lower threshold.

6. The system of claim 2, wherein the controller is further configured to determine whether the measured HRV is above a maximum threshold while or after determining the measured HRV is outside the predetermined range and above the upper threshold.

7. The system of claim 6 further comprising an alarm in electrical communication the controller, and the controller is further configured to set off the alarm upon determining the measured HRV is outside the predetermined range and above the maximum threshold.

8. The system of claim 6, wherein the controller is further configured to control the heat exchanger to warm fluid entering the bladder upon determining the measured HRV is above the upper threshold.

9. The system of claim 6 further comprising a valve configured to open to allow fluid to bypass the heat exchanger, wherein the controller is further configured to control at least one of power delivered to the heat exchanger, an operating state of the valve and a flow rate of the pump to warm fluid entering the bladder upon determining the measured HRV is above the upper threshold.

10. A method for cooling a person, comprising:
pumping fluid through a heat exchanger to a bladder placed on a person;
removing heat from the fluid as the fluid passes through the heat exchanger;
measuring a temperature of the fluid passing through at least one of the heat exchanger and the bladder;
monitoring the person's HRV with an HRV sensor in communication with a controller; and
controlling at least one of power delivered to the heat exchanger and flow of fluid through the heat exchanger based on signals received from the HRV sensor.

11. The method of claim 10, further comprising determining whether a measured HRV is outside a predetermined range defined by a lower threshold and an upper threshold.

12. The method of claim 11, further comprising determining whether the measured HRV is below a minimum threshold while or after determining whether the measured HRV is outside the predetermined range.

13. The method of claim 12, further comprising setting off an alarm upon determining the measured HRV is below the minimum threshold.

14. The method of claim 12, further comprising controlling the heat exchanger to further cool fluid entering the bladder upon determining the measured HRV is below the lower threshold.

15. The method of claim 11, further comprising determining whether the measured HRV is above a maximum threshold while or after determining whether the measured HRV is outside the predetermined range.

16. The method of claim 15, further comprising setting off an alarm upon determining the measured HRV is above the maximum threshold.

17. The method of claim 15, further comprising controlling the heat exchanger to warm fluid entering the bladder upon determining the measured HRV is above the upper threshold of the predetermined range.

18. The method of claim 15, further comprising controlling at least one of the heat exchanger and a valve to bypass the heat exchanger to warm fluid entering the bladder upon determining the measured HRV is above the upper threshold of the predetermined range.
